(19) United States
(12) Patent Application Publication
(10) Pub. No.: US 2026/0203686 A1
(43) Pub. Date: Jul. 16, 2026

(54) Integrated Hardware-Software Architecture for Distributed Resource Monitoring and Real-Time Data Synchronization

(71) Applicant: Maxwell McIntosh, Puyallup, WA (US)

(72) Inventor: Maxwell McIntosh, Puyallup, WA (US)

(21) Appl. No.: 19/434,836

(22) Filed: Dec. 29, 2025

Related U.S. Application Data
(60) Provisional application No. 63/746,095, filed on Jan. 16, 2025.

(51) Int. Cl. G06Q 10/0631; G06Q 10/04; G16H 40/20; G16H 40/40; G16Y 10/60; G16Y 20/10; G16Y 40/10

(52) U.S. Cl. CPC G06Q 10/063114; G06Q 10/04; G16H 40/20; G16H 40/40; G16Y 10/60; G16Y 20/10; G16Y 40/10

(57) ABSTRACT

A distributed system for real-time resource telemetry and synchronized data processing. The system includes a physical monitoring layer with RFID and IoT sensors that track the state of physical assets in a localized network. An integration layer synchronizes sensor telemetry with external clinical databases via an API gateway. A centralized processing node executes a genetic algorithm and machine learning models to calculate optimal resource allocation matrices and resolve data overlaps. Multiple distributed terminal interfaces provide role-optimized access to real-time state maps, ensuring high-fidelity data integrity across the network.

### CROSS-REFERENCE TO RELATED APPLICATIONS

This application claims the benefit of U.S. Provisional Patent Application No. 63/746,095 , filed on Jan. 16, 2025, the entire specification of which is incorporated herein by reference in its entirety.

### BACKGROUND

### Field of the Art

The present disclosure relates generally to the field of distributed computing and automated sensor-integrated resource allocation. More specifically, the disclosure provides systems and methods for real-time telemetry processing, multi-interface data synchronization, and predictive resource state optimization within a localized network environment.

### Discussion of the State of the Art

In high-stakes localized network environments, the dynamic allocation of physical assets and personnel remains a significant computational challenge due to the heterogeneity of connected devices and the multidimensionality of real-time data. Traditional systems for resource state tracking often rely on fragmented software tools and disjointed, non-synchronized records, which lead to latency in data fusion and high error potential in distributed state updates.

### SUMMARY

The present disclosure provides a distributed system and method for real-time resource telemetry and automated synchronization in a localized network environment. The system utilizes a multi-layer hardware-software architecture comprising a plurality of RFID-enabled sensors and IoT monitoring nodes configured to transmit real-time state data across an API gateway to a centralized processing engine. By integrating a physical telemetry layer with a high-performance optimization node, the system executes a genetic algorithm to resolve data packet overlaps and resource availability conflicts across multiple distributed terminal interfaces. This technical architecture ensures high-fidelity data integrity between a persistent data layer and external clinical database systems, reducing latency in record synchronization and improving the computational efficiency of automated resource allocation.

There exists a critical need for an integrated, automated, and sensor-driven architecture capable of high-speed data processing, predictive state modeling, and real-time synchronization across distributed interfaces to mitigate data collisions and optimize system-wide resource efficiency.

### BRIEF DESCRIPTION OF THE DRAWING FIGURES

The accompanying drawings illustrate several aspects and, together with the description, serve to explain the principles of the invention according to the aspects. It will be appreciated by one skilled in the art that the particular arrangements illustrated in the drawings are merely exemplary, and are not to be considered as limiting of the scope of the invention or the claims herein in any way.

FIG. 1 is a block diagram illustrating an exemplary overall system architecture with detailed component interactions and data flow pathways.

FIG. 2 is a block diagram showing the frontend layer components

FIG. 3 depicts the surgeon interface featuring an interactive facility map with real-time operating room status, equipment and staff monitoring, and procedure scheduling capabilities.

FIG. 4 depicts the patient portal interface displaying surgery information, care team details, location information, and pre-surgery requirements with automated notification features.

FIG. 5 depicts the front desk interface with navigation panel, read-only surgery schedule, and patient status tracking.

FIG. 6 Is a system integration diagram illustrating EMR integration components.

FIG. 7 Depicts the real-time resource tracking architecture showing IoT and RFID components.

FIG. 8 Illustrates an AI optimization engine workflow including input data sources, procedure analysis, machine learning models, genetic algorithms, and conflict resolution components.

FIG. 9 is a flowchart showing the surgeon scheduling workflow from login through booking.

FIG. 10 is a flowchart of a genetic algorithm optimization process for operating room scheduling.

FIG. 11 illustrates a rule-based conflict detection and resolution engine workflow showing detection and solving of scheduling conflicts.

FIG. 12 is a flow chart of a machine learning based duration prediction process for estimating procedure length.

FIG. 13 is a block diagram illustrating an exemplary hardware architecture of a computing device showing processors, memory, storage, network interface, and input/output interfaces connected via a common bus or interconnect.

FIG. 14 is a block diagram illustrating an exemplary distributed system architecture showing client devices, application servers, data storage systems, and external services communicating through one or more networks.

### DETAILED DESCRIPTION

### Definitions

For the purposes of this disclosure, a localized network environment may be understood to encompass a healthcare facility, hospital, surgical center, or clinical environment. Physical assets within such an environment include, but are not limited to, operating rooms, medical equipment such as endoscopes, surgical instruments, and consumable supplies. Personnel as described herein include surgeons, anesthesiologists, nursing staff, surgical technicians, and administrative staff. The term resource-state matrix refers to a surgical schedule, operating room booking, or clinical procedure timeline. Telemetry data encompasses real-time status updates including RFID location pings from equipment or personnel badges, IoT environmental sensor readings regarding room conditions or sterilization status, and synchronization updates from integrated clinical database systems. Record synchronization refers to the automated updating of external clinical databases, such as electronic medical records (EMR) including systems like EPIC or Cerner, to maintain high-fidelity data integrity across the network. A distributed terminal interface may include a surgeon portal, patient interface, administrative dashboard, or operating room control room display. Finally, conflict-free allocation describes a system-calculated state wherein no two procedures are assigned to the same physical assets or personnel simultaneously, and all required hardware is verified through the telemetry layer as sterilized and available.

### Detailed Description of Embodiments and Aspects

The present disclosure provides a distributed computing architecture comprising a multi-layer stack for high-frequency telemetry processing and synchronized record management. At the physical layer, the system incorporates a mesh of heterogeneous sensors, including RFID transceivers and IoT environmental monitoring nodes, which generate a continuous stream of state-data packets. These packets are processed by a localized integration gateway configured to resolve data collisions and perform real-time fusion of sensor telemetry with a persistent database layer. The centralized processing node executes a multi-objective optimization algorithm (e.g., a genetic algorithm) to calculate optimal resource-state matrices, ensuring high-fidelity data integrity and reduced latency across a plurality of distributed terminal interfaces.

To further illustrate the technical principles of the distributed architecture described above, an exemplary implementation in a clinical resource environment is provided below. In this specific embodiment, the “localized network environment” corresponds to a surgical facility, the “physical assets” correspond to medical equipment and personnel, and the “resource-state matrix” corresponds to an operating room schedule. While the following description utilizes healthcare-specific terminology for clarity of illustration, it should be understood that the underlying technical systems for data synchronization, sensor telemetry processing, and algorithmic optimization are applicable to any high-utilization resource environment.

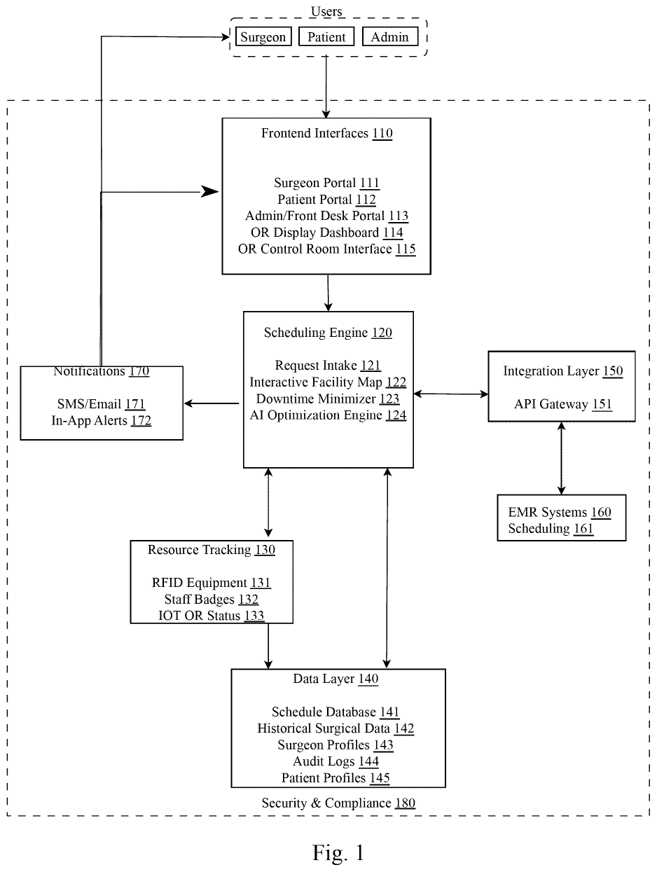

Accordingly, the inventor has conceived and reduced to practice, a system that operates on a scalable and secure architecture, as depicted in FIG. 1. The system is designed around a multi-layer structure that integrates frontend interfaces, a scheduling engine, real-time resource tracking, persistent data storage, and external system integration, all operating under unified security and compliance controls. In the illustrated implementation, FIG. 1 shows the overall system architecture, with components grouped into various functional layers. At the top level, the system accommodates several categories of users, including surgeons, patients, and front desk or administrative staff. Each user interacts with the system through frontend interfaces 110, which typically are role-specific. In the shown implementation, the frontend interfaces include a surgeon portal 111, a patient portal 112, an administrative/front desk portal 113, an operating room display dashboard 114, and an OR Control Room Interface 115. The scheduling engine 120 represents the core processing component, it handles the request intake 121, supports an interactive facility map 122, executes a downtime minimizer 123 to reduce idle periods, and incorporates an AI optimization engine 124 to generate scheduling recommendations. In the illustrated embodiment, real time resource tracking 130 represents an important component of the system architecture, utilizing RFID technology for equipment 131 and staff badges 132, and IoT sensors for OR conditions and availability 133. As shown, all operational data is maintained in the data layer 140. This includes a schedule database 141, historical surgical data 142 to inform predictive models, surgeon profiles 143 containing individual preferences and performance data, audit logs 144 for compliance and system oversight, and patient profiles 145.

The illustrated system architecture also supports external integration through an integration layer 150, which may include an API Gateway 151. This layer communicates with external systems 160, such as EMR systems 161, this API Gateway may also connect to scheduling platforms such as those for the surgeon, nurses, and clinic. This integration allows scheduling data to be synchronized with hospital records as well as external platforms. The system further incorporates notification services 170, which may deliver updates through SMS, MMS, or email 171, or through in-app alerts 172. Finally, security and compliance measures 180, are implemented across all layers, as an example, TLS 1.3 may be used for in-transit encryption and AES-256 for at-rest encryption.

In certain embodiments, the architecture of FIG. 1 may be implemented across distributed computing resources using industry-standard technologies. For example, application servers, may process user inputs, execute scheduling logic, and render interfaces using frameworks such as Node.js or Django. Database servers store schedules, user profiles, and resource data in a relational database like PostgreSQL, with redundancy to ensure reliability. Integration servers facilitate communication with external EMR systems via APIs and protocols like HL7 or FHIR, enabling seamless data exchange. The system supports both cloud-based and on-premises deployment, making it adaptable to various healthcare environments.

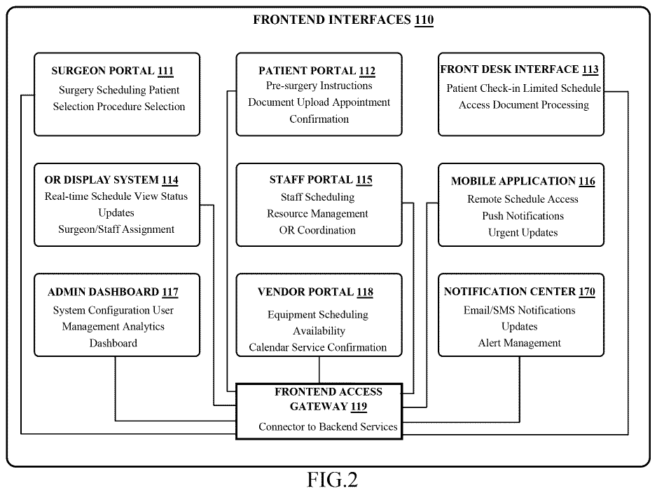

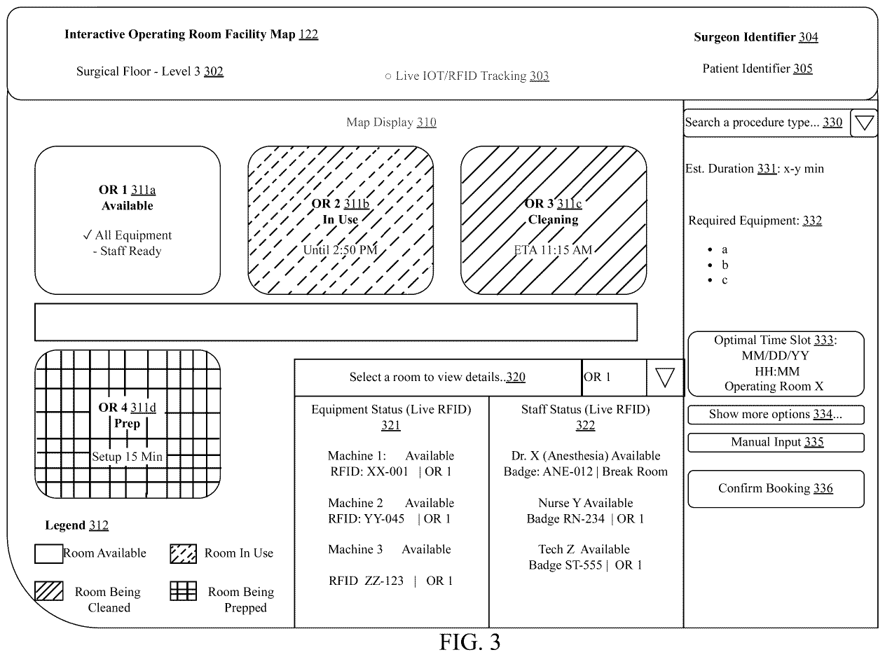

The invention includes multiple user interfaces tailored to different stakeholders as shown in the frontend layer components 110 of FIG. 2. In the illustrated embodiment, the surgeon portal 111, patient portal 112, and front desk interface 113, represent the primary role specific entry points. These provide functions such as surgery scheduling, patient information access, and administrative check-in, with further detail described in FIGS. 3-5. Additional interface components support broader operational functions. An OR display system 114 may provide real-time visualization of operating room status, schedules, and staff assignments for use within clinical environments. A staff portal 115 can facilitate staff scheduling and OR coordination. A mobile application 116 extends system access beyond the hospital network, enabling remote schedule review, push notifications, and urgent updates, often tying in to other portals. An administrative dashboard 117 provides configuration, user management, and analytics functions to authorized system administrators. External parties may access the system through a vendor portal 118, which supports equipment scheduling, availability tracking, and service confirmations for third-party providers. The notification center 170 is also tied directly to the frontend interfaces. All of the illustrated frontend components communicate with backend services via a frontend access gateway 118.

The surgeon interface, shown in FIG. 3, enhances autonomy and efficiency by providing an interactive facility map 122 that displays real-time OR availability with status indicators. In the illustrated embodiment, when the surgeon logs in to the interface, the system displays an informational section with contextual information such as the selected floor or hospital section 302 and confirmation of live IoT and RFID tracking status 303. The surgeon's name appears in a clickable element 304, which when selected provides access to settings and logout options. A patient selection field 305 enables the surgeon to identify the specific patient for whom the procedure is being scheduled, with patient data retrieved from integrated EMR systems after selection.

As illustrated, individual operating rooms 311a-d may have their status represented with distinct visual patterns, shown in the FIG. 3 using different visual patterns such as solid, hatched, and grid design for illustration purposes, though in typical embodiments would be implemented as color-coded status indicators (e.g., green for available, red for in use, blue for cleaning, yellow for prep). A legend 312 provides clear identification of what each visual indicator represents. Surgeons interact with the facility map through direct visual interface interactions, such as clicking or touching the visual representations of operating rooms to view details. Once a room is selected, the system displays its details 320, including live equipment status 321, utilizing RFID tags to display the availability of all equipment assigned to the room, as well as staff status 322, which is monitored through RFID badges.

A procedure selection interface 330 with both dropdown and search functionality allows surgeons to select a procedure type, such as “Laparoscopic Cholecystectomy,” which automatically generates and displays estimated durations 331 and required equipment 332. In some embodiments, the procedure selection interface 330 includes a priority classification, to determine the procedure as either emergency, urgent, or elective. This classification is often built into the searchable dropdown itself, for example, a surgeon may select “Cholecystectomy—Emergency” as opposed to “Cholecystectomy—Elective”. When included, this additional classification allows the AI optimization engine 124 to factor in urgency to scheduling recommendations. Once a procedure is selected, the AI optimization engine 124 suggests an optimal OR and time slot 333 based on AI predictions of procedure requirements, surgeon preferences, and real time resource availability. In the shown embodiment, the surgeon has the option to show more options 334 for alternate suggestions, or do a manual input 335 to make adjustments. If the surgeon selects to confirm the booking 336, the confirmed information is instantly synchronized with the other interfaces as well as external EMR systems.

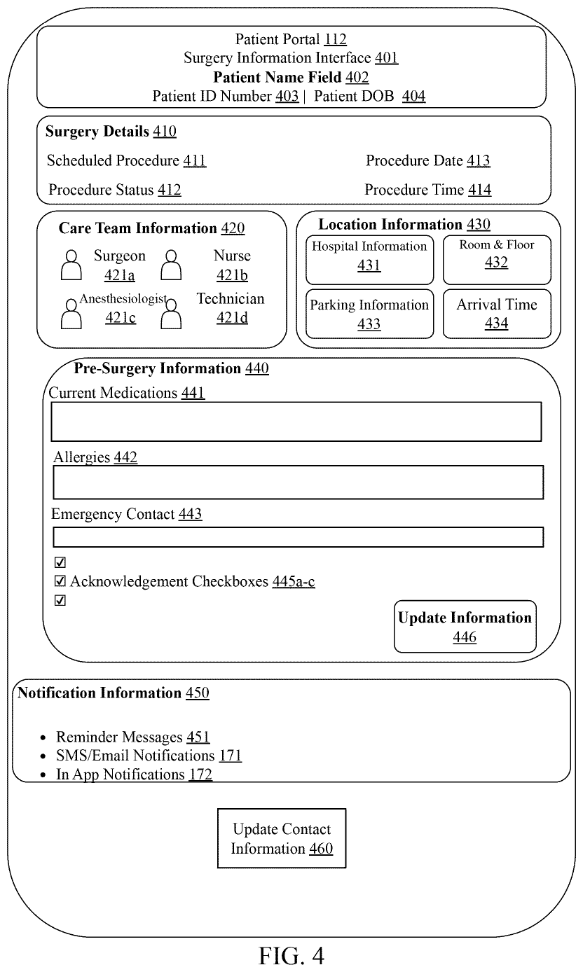

The patient interface, one possible embodiment of which is illustrated in FIG. 4, automatically updates with the scheduled procedure information, improving engagement by providing comprehensive surgery information through a structured portal interface. The shown embodiment of the patient portal 112 includes an interface of surgery information 401 which may include sections for patient name 402, Patient ID 403, and Date of Birth 404. The patient interface improves engagement by displaying surgery details, including date, time, location, and care team information, post-booking. A surgery details display 410, typically includes the scheduled procedure name 411, confirmation status 412, procedure date 413 and time 414 in an easily readable and accessible format. In some embodiments, this section further details educational and preparatory information for the scheduled procedure, which may include, but is not limited to, a general description of the procedure, indicative recovery timelines, examples of products or devices that may be used, pre-operative and post-operative expectations, illustrative diagrams outlining procedural stages, and a 3D animation or video depicting a generalized version of the procedure. A care team information section 420 displays for the patient all staff working on their surgical team, including individual profiles showing staff which include, but is not limited to the surgeons 421a, nursing staff 421b, anesthesiologist 421c, and surgical technicians 421d. Several embodiments of this interface include options to display multiple individuals in each category or role. Location information 430 provides practical details including hospital information 431, specific operating room assignment 432, parking instructions 433, and required arrival time 434 to optimize patient preparation.

The interface captures essential pre-surgery information 440 through interactive forms where patients can input required data such as current medications 441, document known allergies 442, and provide emergency contact details 443, among other required documents and information. Patients must complete pre-operative acknowledgments 445a-445c which may include dietary restrictions, surgical consent confirmations, and post-surgery transportation arrangements, with checkboxes to verify understanding and compliance with hospital protocols. An update mechanism 446 enables patients to submit their information once inputted, or to alter it if needed. The system sends automated reminders 450 to patients, including notifications sent in app 172 or delivered via SMS or Email 171. All patient information is automatically synchronized with the hospital's electronic medical records. The shown embodiment depicts a mobile interface optimized for smartphone and tablet access; however, the patient portal can be accessed through various platforms including desktop computers, mobile devices, and web browsers, ensuring accessibility across different user preferences and technical capabilities.

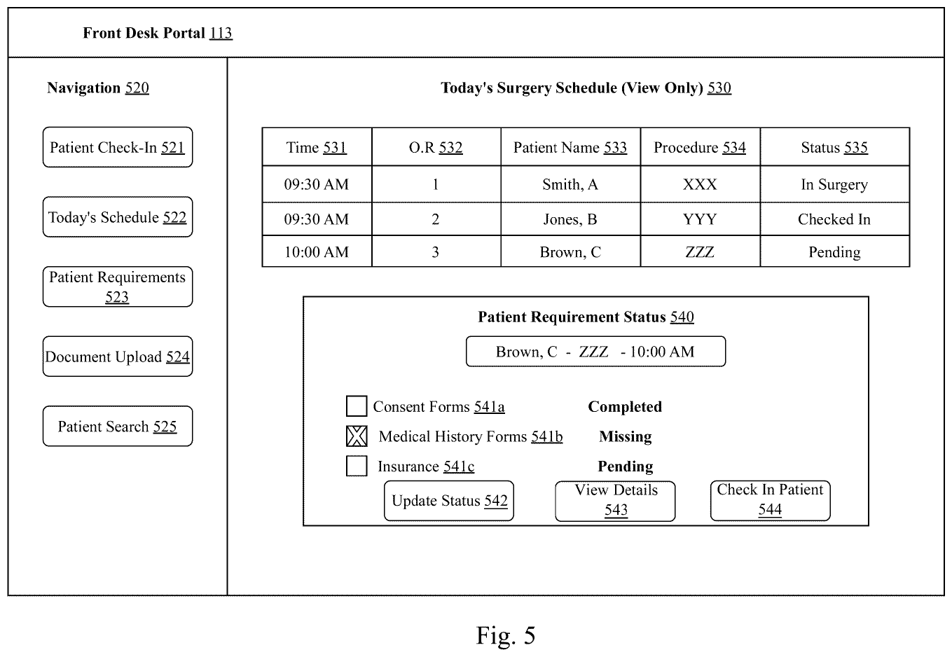

The front desk interface 113, shown in FIG. 5, supports administrative oversight by providing staff with a read-only view of the daily surgery schedule 530 displaying essential information including procedure times 531, operating room assignments 532, patient names 533, scheduled procedures 534, and current status indicators 535. Navigation controls 520 allow front desk personnel to access key functions such as patient check-in 521, daily schedule review 522, patient requirements tracking 523, document upload capabilities 524, and patient search functionality 525. According to one embodiment, the schedule may include color coding, such as green and red, to indicate availability of the operating room. The system displays real-time patient requirement status 540 for upcoming procedures, showing completion progress for items such as consent forms, medical history documentation, and insurance verification. Status indicators are given to the various items, typically either completed, missing, or pending to allow for staff to address outstanding items prior to patient check in. Options to update the form status 542, access a detail view 543 of pending information (which may include, for example, pending insurance items together with insurance company contact information that front desk personnel can use to contact the insurer and complete outstanding requirements), or check the patient in 544 represent some of the very limited modification capabilities granted to front desk personnel to maintain security while preventing unauthorized changes to the surgical schedule.

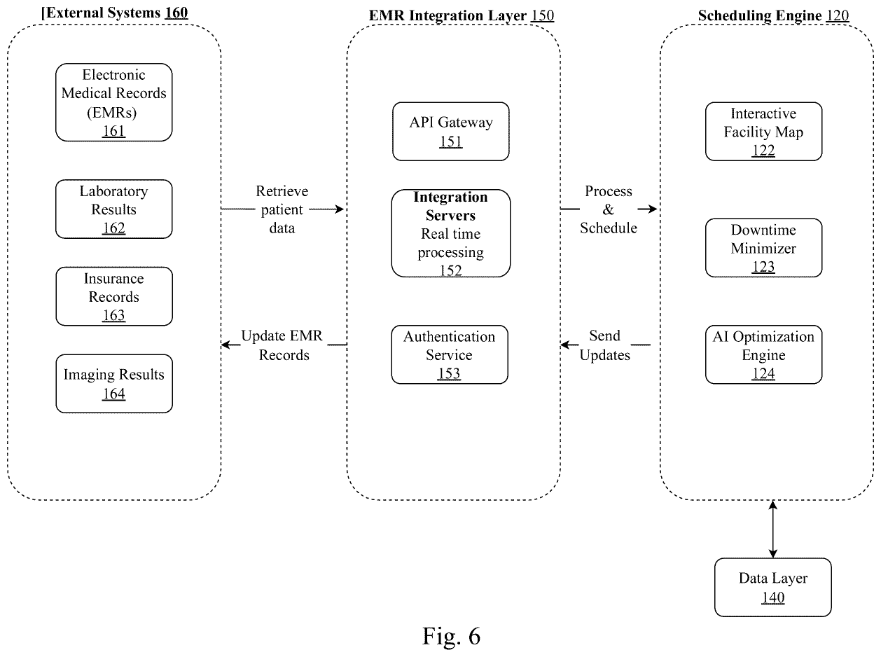

Integration with EMR systems is a key component, as illustrated in FIG. 6. In the illustrated embodiment, the platform integrates multiple external systems 160, including EMRs 161 such as EPIC and Cerner, which contain patient histories and clinical data. Other external sources may include laboratory results 162, insurance records 163, and imaging results 164. Communication between the external systems and the OR scheduling engine 120 is managed through an EMR integration layer 150, which utilizes dedicated integration servers 152 with real time data processing capabilities. An API gateway 151 ensures secure communications by utilizing industry standard protocols such as HL7 or FHIR. An authentication service 153 typically validates data integrity and authenticity from external sources. The OR scheduling engine system 120 retrieves patient data from the integration layer 150 to incorporate its scheduling decisions through the interactive facility map 122, downtime minimizer 123, and AI optimization engine 124, and stored in the OR database 140. Confirmed schedules and booking updates are pushed back through the integration layer 150 to synchronize with the external systems 160.

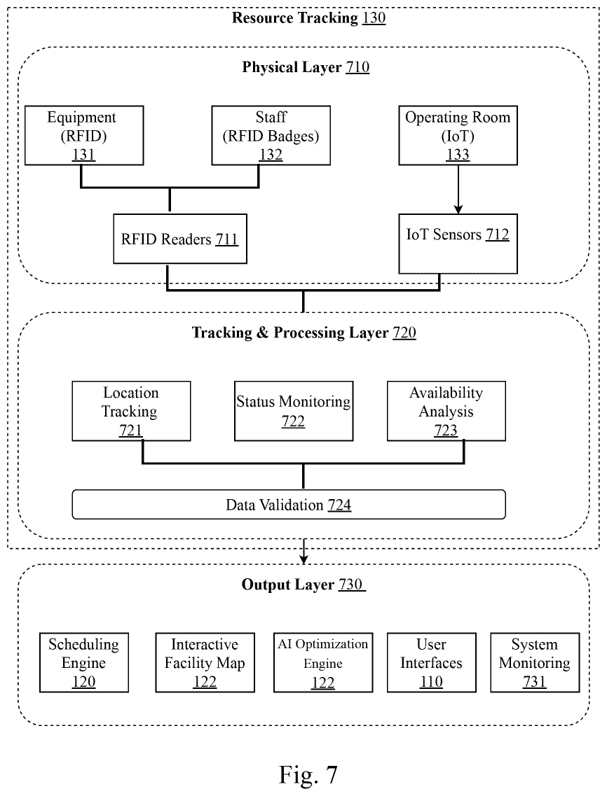

The system also incorporates real-time resource tracking 130, as depicted in FIG. 7. The physical layer 710 monitors equipment 131 such as endoscopes and surgical equipment through RFID tags, staff 132 through RFID-enabled badges, and operating room conditions 133 through IoT devices. Data from the RFID tags and badges is read by RFID readers 711 which are positioned throughout the facility, while IoT sensors 712 collect data from the operating room monitoring systems 133 to track sterilization status and environmental conditions. This information from the physical layer 710 is then processed through the data processing layer 720, which in the illustrated embodiment includes location tracking 721, status monitoring 722, and availability monitoring 723, with the accuracy being ensured through data validation 724. Once confirmed, the processed data is output to external systems 730, including the interactive facility map 122, the scheduling engine 121 and AI optimization engine 124, the various user interfaces 110, and system monitoring 731, which may include functions such as providing alerts for equipment leaving the premises or unauthorized access, tracking overall system performance and monitor sensor health and battery levels.

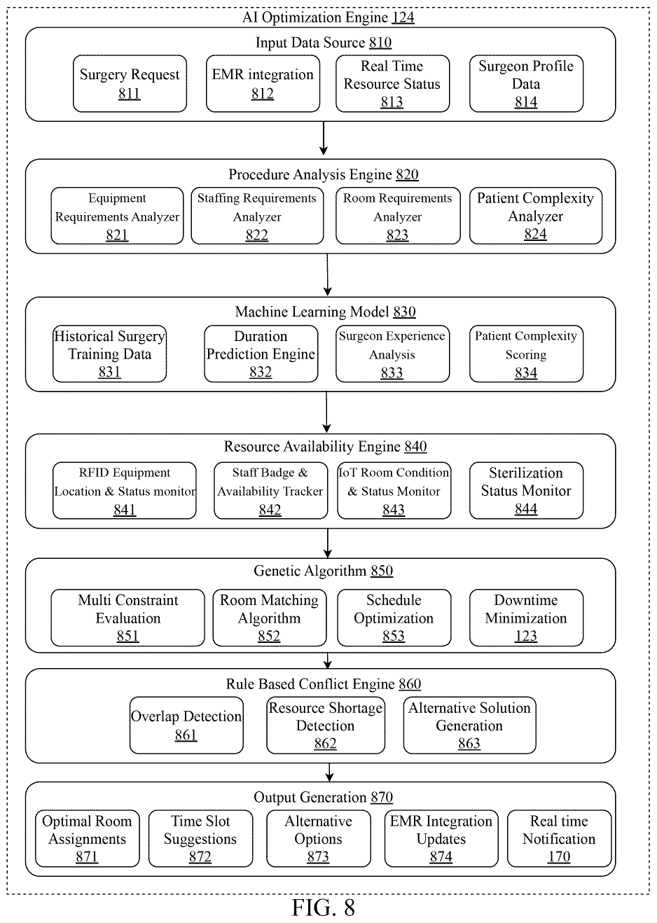

AI-driven scheduling is a central feature of the system, as outlined in FIG. 8. The system incorporates several input data sources 810 into the AI optimization engine 124. The primary input factor is the surgery request 811, specifying the procedure type and patient details. This is combined with EMR integration 812, to automatically pull information such as patient medical histories and test results, real time resource status 813, indicating current equipment and staff availability, as well as a surgeon profile 814, which contains information such as the individual surgeon's preferences and historical performance metrics. In some embodiments, the system may receive input data from any third-party patient-care software capable of communicating through the platform's open API, allowing such external systems to supply clinical, scheduling, or operational information directly to the AI optimization engine. The input data 810 is then entered into the procedure analysis engine 820, which evaluates multiple factors such as equipment requirements 821, staffing requirements 822, room requirements 823, and patient complexity 824, which considers factors such as existing medical conditions, age, medical history, and procedure risk levels. This analysis is fed into the machine learning model 830, which processes historical surgery data 831, trained from a significant number of previous procedures to establish baseline patterns and potential outcomes. The model also applies a duration prediction engine 832, to estimate procedure length based on details which include, but are not limited to surgery type, surgeon experience, and patient age and health status, medical history, and equipment requirements. The machine learning model 830 conducts a surgeon experience analysis 833, which evaluates individual performance metrics and scheduling preferences to optimize assignments. Patient complexity scoring 834 analyzes multiple patient health factors to predict details such as potential complications or extended procedure times. All combined aspects of this machine learning model 830 enable more accurate scheduling and resource allocation. The system also incorporates a resource availability engine 840, which continuously monitors real time conditions throughout the entire facility. This includes RFID equipment location & status monitoring 841, which tracks the location and status of all surgical instruments and specialized equipment; staff badge and availability tracking 842 monitors all relevant healthcare personnel through RFID-enabled badges, keeping track of availability and location of staff such as surgeons, anesthesiologists, nurses, and others. IoT room condition and status monitoring 843 uses IoT enabled sensors to track factors such as environmental status of operating rooms and room occupancy status to ensure optimal conditions. Sterilization status monitoring 844 tracks the sterilization status of surgical instruments, equipment, and operating rooms.

Processed data from the machine learning model 830 and resource availability engine 840 is utilized by the genetic algorithm 850, which simultaneously analyzes multiple factors to find the best possible scheduling recommendations. Multi constraint evaluation 851 compares several potentially competing factors such as surgeon preference, equipment availability, patient needs, and staff availability to determine the best solution. The room matching algorithm 852 evaluates this solution along with the specific procedure to find the most suitable operating room. The genetic algorithm 850 then runs a schedule optimization 853 in combination with a downtime minimization process 123, to maximize utilization of facility resources while reducing idle time for operating rooms and staff. Any potential scheduling conflicts or resource shortages identified during the optimization process are run through the rule-based conflict engine 860. Overlap detection 861 detects any potential conflicts in the booking of operating rooms or resources, meanwhile the resource shortage detection 862 detects scenarios in which required equipment or staff may be unavailable. When such conflicts are detected, an alternate solution engine 863 calculates the optimal rescheduling options and resource substitutions in order to mitigate the conflict. The final output generation 870 produces completed scheduling recommendations in multiple forms. This includes optimal room assignments 871, time slot suggestions 872, alternative options 873 in case the primary recommendation is declined, and EMR integrations 874, which automatically synchronize confirmed schedules across multiple hospital EMR systems. In some embodiments, the system may also expose these generated scheduling outputs to any compatible external patient-care software via the open API, enabling third-party systems to retrieve room assignments, time suggestions, and related updates in real time. Real time notification alerts 170 alert all relevant parties of information such as the confirmed bookings, any schedule changes or booking updates through multiple communication channels.

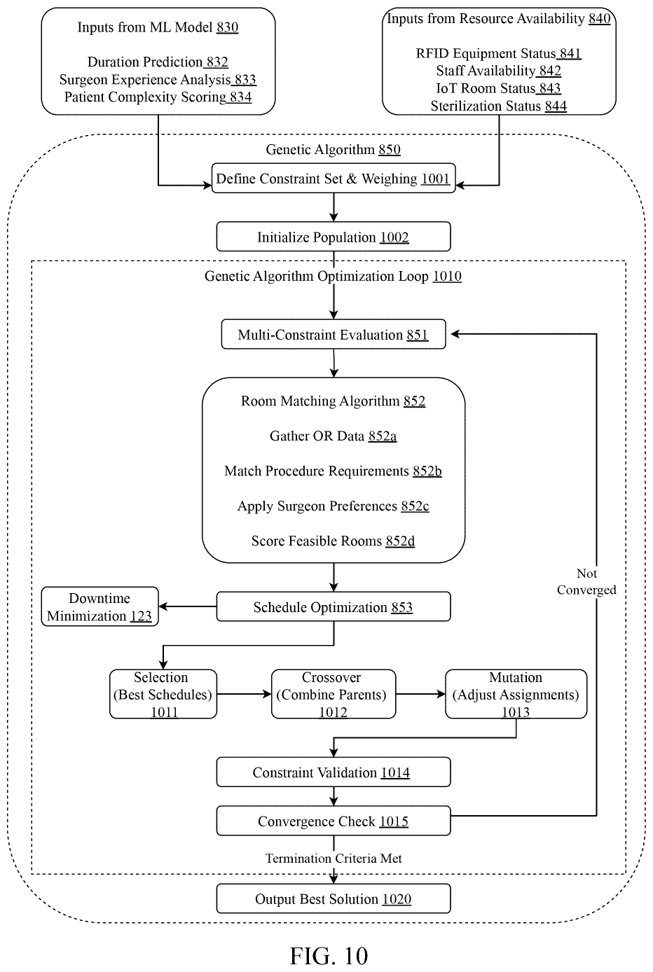

The genetic algorithm 850 of the AI optimization engine 124 is shown in greater detail in FIG. 10. In the shown embodiment, the process begins after receiving inputs from the machine learning model 830 and resource availability engine 840 as shown in FIG. 8. Based on these inputs, the algorithm first defines constraint sets and weightings 1001, assigning relative importance of various scheduling factors. The system then initializes candidate schedules 1002 to create a diverse population of potential solutions. The core processing occurs within the Genetic Algorithm Optimization Loop 1010, which contains several key components. Multi-Constraint Evaluation 851 assesses each candidate schedule against defined criteria. The Room Matching Algorithm 852 performs detailed analysis through four sub-processes, first gathering operating room data 852a to retrieve current operating room information and parameters, matching procedure requirements 852b aligns surgical needs with room capabilities, applying surgeon preferences 852c incorporates individual surgeon requirements and preferences, and scoring of feasible rooms 852d ranks all suitable options.

Following room matching, Schedule Optimization 853 balances assignments across time and resources, while Downtime Minimization 123 works to reduce idle periods between procedures. The algorithm then performs evolutionary operations, including selection of the best candidate schedules 1011, creating offspring solutions through crossover by combining parent schedules 1012, and adjusting assignments through mutation 1013. Each generation undergoes Constraint Validation 1014 to ensure all hard requirements are met. The Convergence Check 1015 evaluates whether termination criteria have been satisfied. The convergence check may be satisfied by various criteria, such as meeting or exceeding a certain fitness score, having gone over a certain number of generations without improvement, or if a maximum number of generations is reached. If none of the criteria are satisfied, the loop continues. Upon meeting termination criteria, the algorithm produces the Output Best Solution 1020.

In some embodiments, the genetic algorithm 850 is implemented using parallel processing techniques, with candidate schedule evaluation distributed across multiple processor cores. The algorithm maintains a diverse population through techniques such as fitness sharing and niching, preventing premature convergence to suboptimal solutions. The constraint validation 1014 employs both hard constraints which must be satisfied and soft constraints which influence fitness scores, enabling flexible optimization that respects critical requirements while maximizing overall schedule quality.

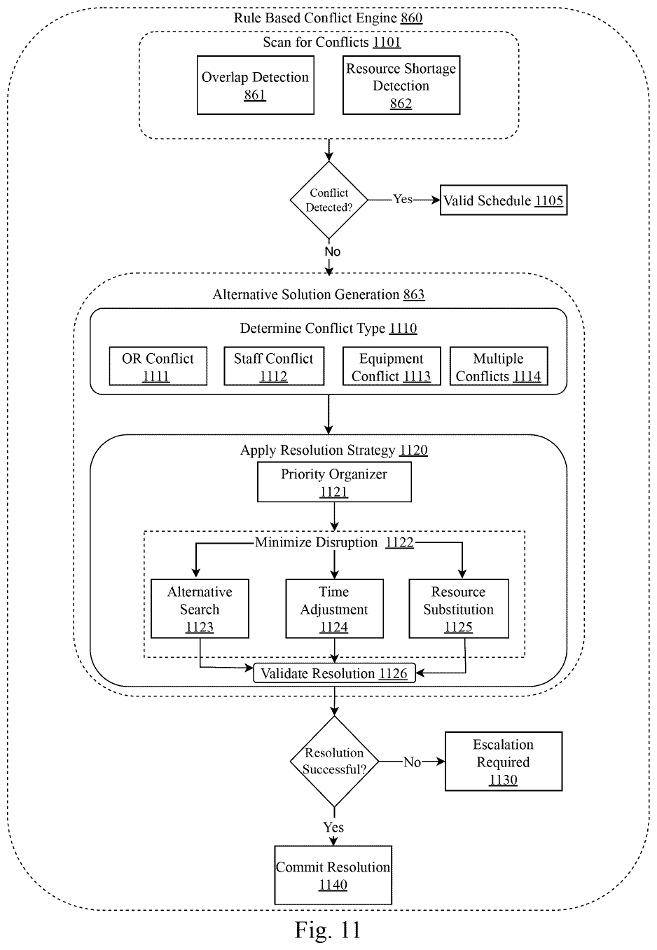

The genetic algorithm 850 results are processed by the rule-based conflict engine 860, illustrated in FIG. 11. In the shown implementation, the process begins with a conflict scan 1101, which includes checks for room overlap detection 861 and resource shortage detection 862. If no conflict is detected, the schedule is marked as a valid schedule 1105 and passed forward to output generation 870. If a conflict is identified, the system initiates an alternative solution generation stage 863. At this stage, the system determines the conflict type 1110. Examples include an OR conflict 1111, where an operating room is double-booked; a staff conflict 1112, where personnel are scheduled for overlapping procedures; an equipment conflict 1113, where a required instrument is either in use elsewhere or not yet sterilized; and multiple concurrent conflicts 1114, where two or more conflict types occur together.

Once the conflict type is established, the system applies a resolution strategy 1120. A priority organizer 1121 may account for urgency classifications such as emergency, urgent, and elective procedures, which are typically selected at the time of procedure selection, and assigns an execution order accordingly. The minimize disruption process 1122 attempts to resolve conflicts with limited changes to the existing schedule. Within this process, an alternative search 1123 evaluates backup operating rooms or staff, a time adjustment 1124 shifts procedure start times, and a resource substitution 1125 identifies equivalent staff or equipment. Each candidate resolution is then validated 1126 to confirm that the conflict has been resolved without creating new issues. If validation is successful, the resolution is committed 1140 and the affected schedules are updated. If validation fails, the conflict is escalated 1130 for supervisory review and manual intervention. As an example, if two procedures would be simultaneously scheduled in the same operating room, the system may resolve the conflict by reallocating one to an alternate available room through alternative search 1123. Were an essential staff member such as an anesthesiologist to be double booked, the system may substitute another qualified anesthesiologist through resource substitution 1125. If there was a scenario where no acceptable alternatives are identified, the system applies a time adjustment 1124, selecting the closest non-conflicting time while preserving as much of the original schedule as possible.

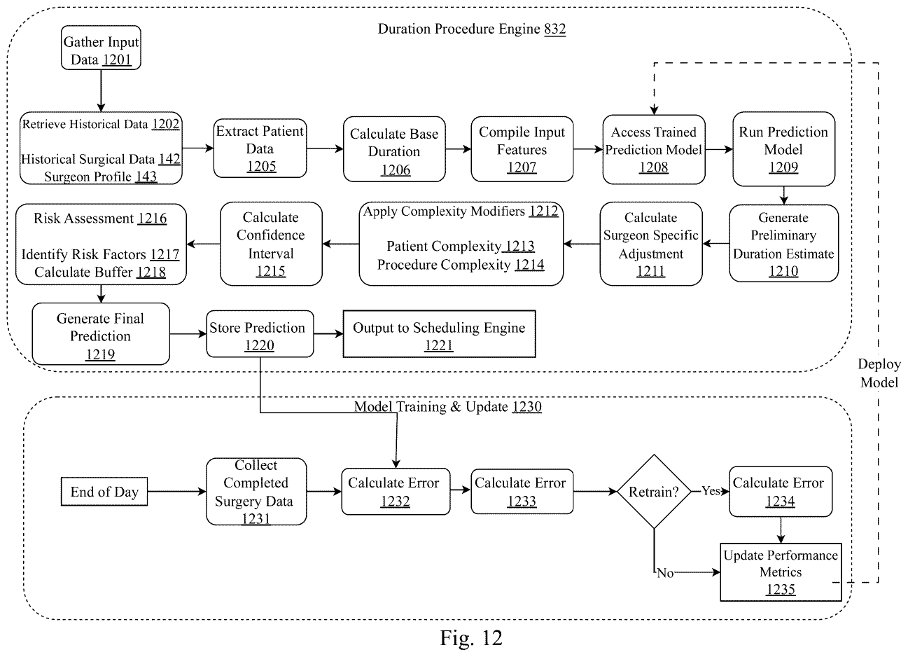

FIG. 12 illustrates in greater depth the duration procedure engine 832, which operates as a subcomponent of the machine learning model 830 within the AI optimization engine 124. The duration procedure engine 832 is configured to estimate surgical procedure durations by integrating historical procedural data with real-time patient-specific variables, while continuously refining its predictions through automated feedback from completed surgical cases. The workflow begins with the gather input data module 1201 and retrieve historical data module 1202, which may include historical surgical data 142 such as completion times and complication rates, as well as surgeon profiles 143 containing efficiency and experience indicators. For new scheduling requests, the system extracts patient data 1205, calculates a base duration 1206, and compiles input features 1207 for use by a trained prediction model 1208. The run prediction model step 1209 generates a preliminary duration estimate 1210. Surgeon specific adjustments 1211 are then made to the estimate, weighing factors such as the surgeon's experience with the procedure and historical performance. The system then further refines this estimate using complexity modifiers 1212, which typically includes patient complexity 1213 and procedure complexity 1214. Patient complexity 1213, which may include factors such as advanced age, obesity, cardiac or respiratory conditions, and surgical history. Procedure complexity 1214, which may include factors such as surgical difficulty, anatomical region, and instrumentation requirements. A risk assessment 1216 is performed, including identification of risk factors 1217 and calculation of a buffer 1218. Together with calculation of a confidence interval 1215, the engine produces a final prediction 1219. This prediction is stored 1220 and transmitted to the scheduling engine 1221 for optimization

The lower portion of FIG. 12 depicts the model training and update process 1230. At the end of each day, the system collects completed surgery data 1231, calculates error values 1232 and 1233, and determines whether retraining criteria are satisfied. If retraining is triggered, the system performs additional error analysis 1234 to validate updated models. Regardless of retraining, performance metrics 1235 are updated to maintain accuracy tracking and trend analysis. This continuous feedback loop enables the duration procedure engine 832 to improve over time, adapting to surgeon-specific performance patterns, evolving procedural practices, and changes in patient populations.

Security and compliance 180 are prioritized throughout the system. Typically, data is protected using TLS 1.3 for in-transit encryption and AES-256 for at-rest encryption. Role-based access controls with multi-factor authentication ensure that only authorized users can access sensitive information. The system also maintains audit logs and undergoes regular security assessments to meet HIPAA compliance requirements.

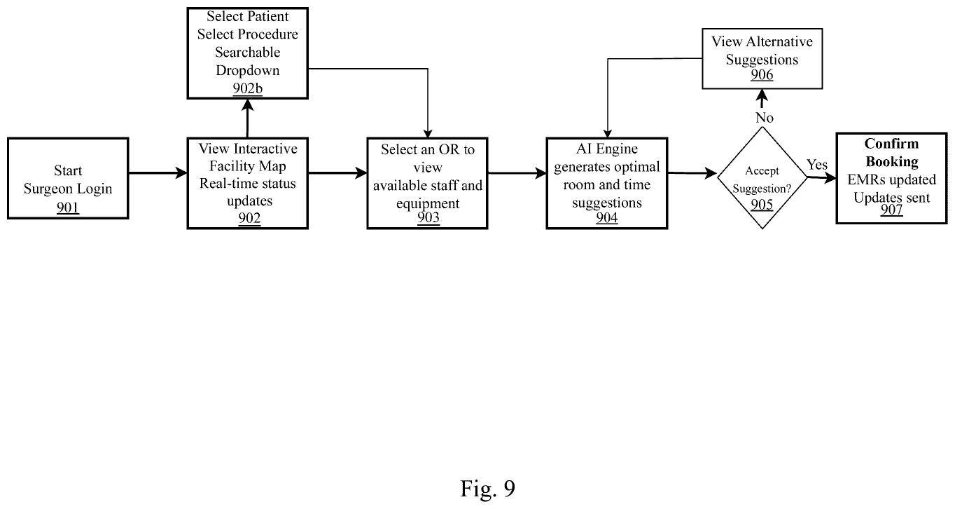

FIG. 9 illustrates the complete scheduling workflow, beginning with surgeon login 901. After login, the system displays the interactive facility map 902 with real-time operating room status. From this screen, the surgeon may select the patient and procedure 902b using a searchable dropdown. The surgeon then selects an operating room 903 to view available staff and equipment. Based on this information, the AI engine generates an optimal room and time suggestion 904. At decision point 905, the surgeon may either accept the suggestion or view alternative options 906. If the suggestion is accepted, the booking 907 is confirmed and updates are sent to the EMR system and connected interfaces. The invention supports several alternative embodiments, including a mobile app for remote scheduling access, voice-activated controls for hands-free operation in sterile environments, and adaptation for outpatient or diagnostic scheduling. These variations broaden the system's applicability across different healthcare settings.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### Hardware Architecture

The techniques disclosed herein may be implemented on hardware, software, or a combination thereof. Implementation may occur on one or more computing devices including but not limited to: servers, personal computers, mobile devices, embedded systems, virtual machines, containerized environments, serverless platforms, edge computing nodes, or distributed computing systems.

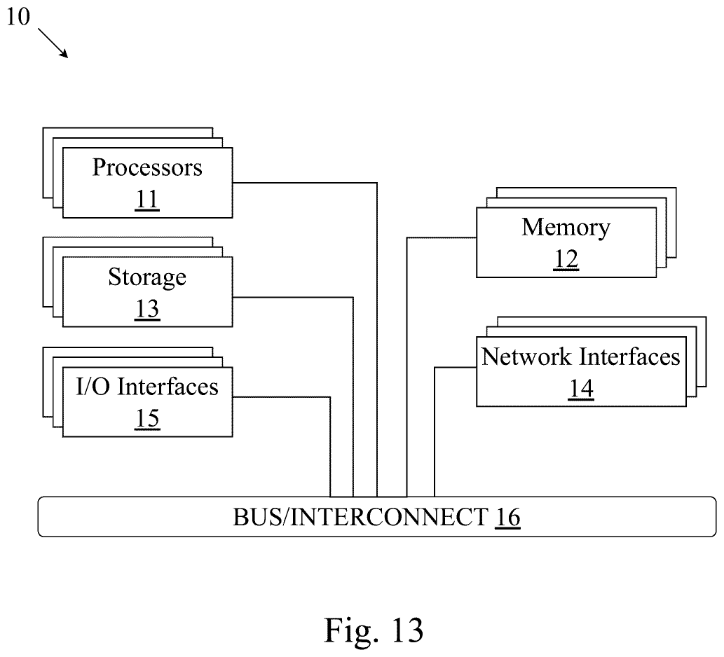

FIG. 13 illustrates an exemplary computing device 10 suitable for implementing the disclosed aspects. The device includes one or more processors 11 (CPUs, GPUs, TPUs, or other processing units), memory 12 (volatile and/or non-volatile), storage 13 (local and/or remote), network interfaces 14, and input/output interfaces 15, connected via one or more communication buses or interconnects 16. The specific hardware configuration may vary without departing from the scope of the invention.

The centralized processing node is implemented via the one or more processors 11 and memory 12 of the computing device 10. Specifically, the hardware processor is configured as a high-throughput telemetry aggregator that establishes a dedicated execution environment for the AI optimization engine 124. The memory 12 stores the persistent state of the resource-allocation matrix, which is continuously updated via the network interface 14 as new data packets are received from the API gateway 151 and the physical monitoring layer 710. This hardware configuration enables the concurrent execution of the genetic algorithm 850 and machine learning models 830, ensuring that complex multi-objective resource constraints are resolved with minimal latency to maintain high-fidelity data integrity across the distributed terminal interfaces 110.

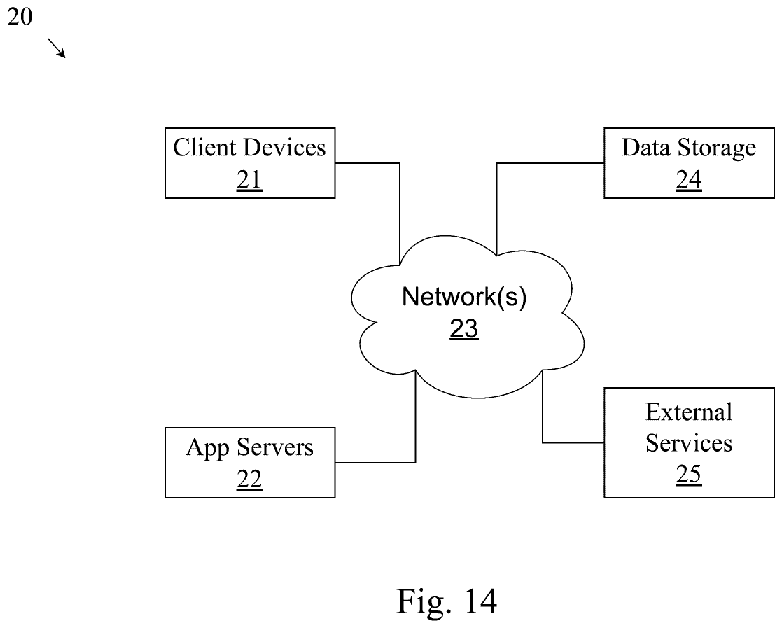

FIG. 14 illustrates an exemplary distributed system architecture 20 for implementing aspects across multiple computing devices. The architecture includes client devices 21, application servers 22, one or more networks 23 (Internet, intranet, cellular, or other), data storage systems 24 (databases, object storage, distributed file systems), and external services 25 (APIs, cloud services, third-party integrations). Components may communicate using standard protocols including but not limited to HTTP/HTTPS, WebSocket, gRPC, message queues, or custom protocols. The system may be deployed on-premise, in public/private clouds, edge locations, or hybrid combinations thereof.

Software components may be deployed as native applications, web applications, mobile applications, containerized microservices, serverless functions, or any combination. Data may be stored in relational databases, NoSQL databases, key-value stores, graph databases, time-series databases, flat files, distributed ledgers, or other storage systems. Security measures may include authentication, authorization, encryption, audit logging, and other standard practices.

The skilled person will recognize that the specific hardware and software configurations described are exemplary. The invention may be implemented on any suitable computing infrastructure, and functionality may be distributed across components in various ways without departing from the scope of the claims.

What is claimed is:

1. A distributed system for automated resource telemetry and synchronized state processing, the system comprising:
a physical monitoring layer comprising a plurality of radio-frequency identification (RFID) sensors and Internet of Things (IoT) environment sensors configured to detect real-time state changes of a plurality of physical assets;
a network integration layer comprising an API gateway configured to establish a persistent bi-directional communication link with at least one external clinical database for real-time record synchronization ; and
a centralized processing node comprising a hardware processor and a non-transitory computer-readable medium storing instructions that, when executed, cause the processor to:
receive telemetry data from the physical monitoring layer and the network integration layer;
transform said telemetry data into a visual state map representing resource availability across a localized network environment; and
execute an artificial intelligence optimization engine comprising a machine learning model and a genetic algorithm to calculate a conflict-free resource allocation matrix based on detected sensor telemetry and synchronized database records.

2. The system of claim 1, wherein the machine learning model is configured to predict a temporal duration for a resource state based on historical telemetry data and specific features of the physical assets.

3. The system of claim 1, wherein the genetic algorithm is configured to resolve multi-objective constraints including asset location, personnel availability, and environmental requirements to generate the resource allocation matrix.

4. The system of claim 1, further comprising a rule-based conflict engine configured to perform overlap detection on the resource allocation matrix and propose alternate asset assignments in real time.

5. The system of claim 1, wherein the localized network environment is an operating room facility, and the resource allocation matrix defines a surgical schedule.

6. The system of claim 1, wherein the API gateway utilizes HL7 or FHIR protocols to synchronize data with an electronic medical record (EMR) system.

7. The system of claim 1, wherein the physical monitoring layer includes RFID-enabled badges for tracking the real-time location and availability status of personnel within the localized network environment.

8. The system of claim 1, wherein the IoT environment sensors monitor at least one of sterilization status, room temperature, or occupancy status.

9. The system of claim 1, further comprising a plurality of role-optimized distributed terminal interfaces configured to display the visual state map with color-coded availability indicators.

10. The system of claim 9, wherein at least one distributed terminal interface is a restricted-access portal providing read-only telemetry views to unauthorized users.

11. A computer-implemented method for automated resource telemetry and synchronized state processing, the method comprising the steps of:
detecting, via a physical monitoring layer comprising RFID and IoT sensors, real-time state changes of physical assets within a localized network environment;
synchronizing, via a network integration layer, real-time telemetry data with an external database through a bi-directional API gateway;
generating a visual state map representing resource availability on a distributed terminal interface; and
calculating, via an artificial intelligence optimization engine, a resource allocation matrix by processing historical state data and real-time sensor telemetry through a machine learning model and a genetic algorithm.

12. The method of claim 11, further comprising predicting a temporal duration for a specific resource allocation using a machine learning model trained on historical performance metrics.

13. The method of claim 11, further comprising applying a genetic algorithm to optimize resource assignments by minimizing downtime between resource state transitions.

14. The method of claim 11, further comprising detecting resource overlaps and proposing automated rescheduling options using a rule-based conflict resolution engine.

15. The method of claim 11, wherein synchronizing data involves pushing confirmed resource allocations back to an external EMR system to maintain global record integrity.

16. The method of claim 11, further comprising transmitting automated notifications to distributed terminals via SMS, email, or in-app alerts upon a change in the resource allocation matrix.

17. The method of claim 11, wherein the visual state map allows for direct interactive selection of localized resources for allocation.

18. The method of claim 11, further comprising implementing role-based access controls and multi-factor authentication for all data interactions within the localized network environment.

19. The method of claim 11, further comprising monitoring the sterilization status of medical equipment via the physical monitoring layer and preventing allocation of non-sterilized equipment within the resource allocation matrix.

20. A resource management system for a clinical environment, comprising:
an interactive facility map displaying real-time availability of surgical operating rooms;
an RFID-based tracking subsystem configured to monitor the location of surgical instruments and personnel badges;
an EMR integration layer for synchronizing patient records and surgical schedules; and
an AI optimization engine configured to:
(i) predict procedure durations based on surgeon historical performance;
(ii) allocate operating rooms using a genetic algorithm; and
(iii) resolve scheduling overlaps via a rule-based engine.
